(19) United States
(12) Patent Application Publication
(10) Pub. No.: US 2013/0171128 A1
(43) Pub. Date: Jul. 4, 2013

(54) REDUCING VISCOSITY OF PHARMACEUTICAL FORMULATIONS

(71) Applicants: Holly Zhuohong Huang, Thousand Oaks, CA (US); Dingjiang Liu, Oak Park, CA (US); Christopher J. Sloey, Newbury Park, CA (US); Camille Gleason, Santa Monica, CA (US)

(72) Inventors: Holly Zhuohong Huang, Thousand Oaks, CA (US); Dingjiang Liu, Oak Park, CA (US); Christopher J. Sloey, Newbury Park, CA (US); Camille Gleason, Santa Monica, CA (US)

(73) Assignee: AMGEN INC., Thousand Oaks, CA (US)

(21) Appl. No.: 13/582,357

(22) PCT Filed: Mar. 1, 2011
(86) PCT No.: PCT/US11/26710

§ 371 Date: Dec. 5, 2012

Related U.S. Application Data
(60) Provisional application No. 61/309,657, filed on Mar. 2, 2010.

(51) Int. Cl. A61K 47/18; A61K 47/20

(52) U.S. Cl. CPC A61K 47/183; A61K 47/20
USPC 424/130.1; 514/1.1

(57) ABSTRACT

A stable pharmaceutical formulation is provided that comprises a biologically active protein and an excipient selected from taurine, theanine, sarcosine, citrulline and betaine.

### CROSS-REFERENCE TO RELATED APPLICATIONS

This application claims the benefit under 35 U.S.C. 119(e) of U.S. patent application No. 61/309,657, filed Mar. 2, 2010, which is incorporated herein by reference.

### BACKGROUND

Pharmaceutically active proteins, such as antibodies, are frequently formulated in liquid solutions, particularly for parenteral injection. For products that need to be administered via a subcutaneous route, for example use in self administration; formulations in delivery volumes greater than 1-2 milliliters are not well tolerated. In such cases highly concentrated protein formulations are desirable to meet the limited dose volume. The high dose and small volume requirements such administration means that the protein therapeutic can reach concentrations of upwards of 100 mg/ml or more. Highly concentrated protein formulations can pose many challenges to the manufacturability and administration of protein therapeutics. One challenge posed by some highly concentrated protein formulations is increased viscosity. High viscosity formulations are difficult to handle during manufacturing, including at the bulk and filling stages. High viscosity formulations are also difficult to draw into a syringe and inject, making administration to the patient difficult and unpleasant. The need to identify compounds that are useful for reducing viscosity of highly concentrated protein formulations, to develop methods of reducing the viscosity of such formulations, and to provide pharmaceutical formulations with reduced viscosity are well known in the pharmaceutical industry. The present invention provides such compounds, methods and formulations.

### SUMMARY

Provided are excipients taurine, theanine, sarcosine, citrulline, betaine and mixtures at selected concentrations for use in reducing the viscosity of protein formulations. Methods for reducing the viscosity of protein formulations by combining a high concentration therapeutic protein with a viscosity-reducing concentration of an excipient selected from the group consisting taurine, theanine, sarcosine, citrulline, betaine and mixtures thereof are provided herein. Also provided is lyophilized powder comprising a therapeutic protein and an excipient selected from the group consisting of taurine, betaine, theanine, citrulline and sarcosine and mixtures thereof, wherein the excipient is present at a weight:weight concentration effective to reduce viscosity upon reconstitution with a diluent.

Provided herein is a method for reducing the viscosity of a liquid pharmaceutical formulation comprising a therapeutic protein at a concentration of at least 70 mg/ml, comprising the step of combining the therapeutic protein with a viscosity-reducing concentration of an excipient selected from the group consisting taurine, theanine, sarcosine, citrulline, betaine and mixtures thereof. In one embodiment the viscosity of the formulation is reduced by at least 5%. In another embodiment the viscosity of the formulation is reduced by at least 30%. In a related embodiment are provided pharmaceutical formulations produced by such methods

Also provided is a pharmaceutical composition comprising a therapeutic protein at a concentration of at least 70 mg/mL, and an excipient selected from the group consisting of taurine, theanine, sarcosine, citrulline, betaine and mixtures thereof. In one embodiment the concentration of the excipient is from about 5 mM to about 700 mM. In a related embodiment the concentration of the excipient is from about 200 mM to about 650 mM. Also provided are such pharmaceutical compositions having a pH between about 4.0 to about 6.0. In a related embodiment the pH is about 4.6 to about 5.2.

Also provided is a method of preparing a lyophilized powder comprising the step of lyophilizing a pharmaceutical formulation as described above.

Provided herein is a lyophilized powder comprising a therapeutic protein and an excipient selected from the group consisting of taurine, theanine, sarcosine, citrulline, betaine and mixtures thereof, wherein the excipient is present at a weight:weight concentration effective to reduce viscosity upon reconstitution with a diluent. In one embodiment the excipient is present at a concentration of between about 100 μg per mg therapeutic protein to about 1 mg per mg therapeutic protein. In a related embodiment the excipient is present at a concentration between about 200 μg to about 500 μg per mg therapeutic protein. Also provided is a method for reconstituting a lyophilized powder as described above comprising the step of adding a sterile aqueous diluent.

Also provided are therapeutic proteins that are antibodies. Also provided are formulations or compositions as described above wherein the therapeutic protein is an antibody. In addition, also provided herein is a lyophilized powder as described above wherein the therapeutic protein is an antibody.

### BRIEF DESCRIPTION OF THE DRAWINGS

FIG. 1 Shows the effect of various excipients at 200 mM on the viscosity of a concentrated antibody formulation.

FIG. 2 Shows the effect of increasing the concentration of citrulline or theanine on the viscosity of a concentrated antibody formulation.

FIG. 3 Shows the effect of various excipients on temperature induced aggregation of a concentrated antibody formulation.

FIG. 4 Shows the effect of L-citrulline vs sucrose on the viscosity of a concentrated antibody formulation.

FIG. 5 Shows the effect of taurine vs sucrose on the viscosity of a concentrated antibody formulation.

FIG. 6 Shows the effect of taurine and sarcosine on thermally induced aggregation of a concentrated antibody formulation.

FIG. 7a Shows the effect of taurine and sarcosine on the viscosity of a concentrated antibody formulation.

FIG. 7B Shows the impact of formulation pH on the viscosity of concentrated antibody formulations containing various excipients

FIG. 8 Shows the effect of taurine vs creatinine or carnitine on the viscosity of a concentrated antibody formulation

### DETAILED DESCRIPTION

Reducing the viscosity of high concentration therapeutic protein formulations is of interest to the pharmaceutical industry. Taurine, betaine, theanine, citrulline and sarcosine were discovered to reduce the viscosity of such formulations. The invention provides such excipients at selected concentrations for use in reducing the viscosity of protein formulations. Methods for reducing the viscosity of protein formulations by combining the therapeutic protein with a viscosity-reducing concentration of an excipient selected from the group consisting taurine, theanine, sarcosine, citrulline, betaine and mixtures thereof are provided herein. Also provided is lyophilized powder comprising a therapeutic protein and an excipient selected from the group consisting of taurine, betaine, theanine, citrulline and sarcosine and mixtures thereof, wherein the excipient is present at a weight:weight concentration effective to reduce viscosity upon reconstitution with a diluent.

Unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990). Enzymatic reactions and purification techniques are performed according to manufacturers specifications, as commonly accomplished in the art or as described herein. The terminology used in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

All patents and other publications identified are expressly incorporated herein by reference in their entirety for the purpose of describing and disclosing, for example, the methodologies described in such publications that might be used in connection with the described.

Zwitterions are characterized as having separate positive and negative charges that result in a net zero charge for the compound. Most amino acids are zwitterions at physiological pH. Zwitterions are useful as surfactants and osmolytes. As disclosed herein, pharmaceutical formulations containing zwitterions, in particular taurine, theanine, sarcosine, betaine and citrulline, were found to generally have lower viscosity than polyol containing formulations while having greater or comparable stability.

Taurine (also known as 2-aminoethanesulfonic acid) is a cysteine derivative and a naturally occurring component of bile and can be found in various tissues. Theanine (also known as gamma-glutamylethylamide, or 5-N-ethyl-glutamine) is a glutamic acid analog. Sarcosine is the N-methyl derivative of glycine. Betaine, (also known as trimethylglycine) is generally known for its osmoprotective properties. Citrulline (2-Amino-5-(carbamoylamino)pentanoic acid) is an intermediate in the urea cycle.

The terms “polypeptide” and “protein” are used interchangeably herein. Exemplary polypeptides contemplated for use in the stable pharmaceutical formulations of the invention include antibodies, peptibodies, immunoglobulin-like proteins, non-antibody proteins and non-immunoglobulin-like proteins. Analogs of naturally occurring proteins are contemplated for inclusion in formulations of the present invention, including polypeptides with modified glycosylation, polypeptides without glycosylation (unglycosylated). As used herein, “analogs” refers to an amino acid sequence that has insertions, deletions or substitutions relative to the parent sequence, while still substantially maintaining the biological activity of the parent sequence, as determined using biological assays known to one of skill in the art. The formulations of the invention may also include derivatives of naturally occurring or analog polypeptides which have been chemically modified, for example, to attach water soluble polymers (e.g., pegylated), radionuclides, or other diagnostic or targeting or therapeutic moieties.

Antibodies may be formulated according to the present invention. As used herein, the term “antibody” includes fully assembled antibodies, monoclonal antibodies (including human, humanized or chimeric antibodies), polyclonal antibodies, multispecific antibodies (e.g., bispecific antibodies), maxibody, and antibody fragments that can bind antigen (e.g., Fab′, F′(ab)2, Fv, single chain antibodies, diabodies), comprising complementarity determining regions (CDRs) of the foregoing as long as they exhibit the desired biological activity.

Peptibodies, molecules comprising an antibody Fc domain attached to at least one antigen-binding peptide, are generally described in PCT publication WO 00/24782. Immunoglobulin-like proteins, members of the immunoglobulin superfamily, contain one or more immunoglobulin-like domains which fold in structures similar to portions of the antibody variable region.

Proteins, including those that bind to one or more of the following, would be useful in the compositions and methods of the present invention. These include CD proteins including, but not limited to, CD3, CD4, CD8, CD19, CD20, CD22, CD30, and CD34; including those that interfere with receptor binding. HER receptor family proteins, including HER2, HER3, HER4, and the EGF receptor. Cell adhesion molecules, for example, LFA-I, Mol, pI50, 95, VLA-4, ICAM-I, VCAM, and alpha v/beta 3 integrin. Growth factors, including but not limited to, vascular endothelial growth factor (“VEGF”), growth hormone, thyroid stimulating hormone, follicle stimulating hormone, luteinizing hormone, growth hormone releasing factor, parathyroid hormone, mullerian-inhibiting substance, human macrophage inflammatory protein (MIP-I-alpha), erythropoietin (EPO), nerve growth factor, such as NGF-beta, platelet-derived growth factor (PDGF), fibroblast growth factors, including, for instance, aFGF and bFGF, epidermal growth factor (EGF), transforming growth factors (TGF), including, among others, TGF-α and TGF-β, including TGF-βI, TGF-β2, TGF-β3, TGF-β4, or TGF-β5, insulin-like growth factors-I and -II (IGF-I and IGF-II), des(I-3)-IGF-I (brain IGF-I), and osteoinductive factors.

Insulins and insulin-related proteins, including but not limited to insulin, insulin A-chain, insulin B-chain, proinsulin, and insulin-like growth factor binding proteins. Coagulation and coagulation-related proteins, such as, among others, factor VIII, tissue factor, von Willebrands factor, protein C, alpha-1-antitrypsin, plasminogen activators, such as urokinase and tissue plasminogen activator (“t-PA”), bombazine, thrombin, and thrombopoietin; (vii) other blood and serum proteins, including but not limited to albumin, IgE, and blood group antigens. Colony stimulating factors and receptors thereof, including the following, among others, M-CSF, GM-CSF, and G-CSF, and receptors thereof, such as CSF-1 receptor (c-fms). Receptors and receptor-associated proteins, including, for example, flk2/flt3 receptor, obesity (OB) receptor, growth hormone receptors, thrombopoietin receptors (“TPO-R,” “c-mpl”), glucagon receptors, interleukin receptors, interferon receptors, T-cell receptors, stem cell factor receptors, such as c-Kit, and other receptors listed herein. Receptor ligands, including, for example, OX40L, the ligand for the OX40 receptor. Neurotrophic factors, including but not limited to, bone-derived neurotrophic factor (BDNF) and neurotrophin-3, -4, -5, or -6 (NT-3, NT-4, NT-5, or NT-6). Relaxin A-chain, relaxin B-chain, and prorelaxin; interferons and interferon receptors, including for example, interferon-α, -β, and -γ, and their receptors. Interleukins and interleukin receptors, including but not limited to IL-I to IL-33 and IL-I to IL-33 receptors, such as the IL-8 receptor, among others. Viral antigens, including but not limited to, an AIDS envelope viral antigen. Lipoproteins, calcitonin, glucagon, atrial natriuretic factor, lung surfactant, tumor necrosis factor-alpha and -beta, enkephalinase, RANTES (regulated on activation normally T-cell expressed and secreted), mouse gonadotropin-associated peptide, DNAse, inhibin, and activin. Integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, addressins, regulatory proteins, immunoadhesins, antibodies. Myostatins, TALL proteins, including TALL-1, amyloid proteins, including but not limited to amyloid-beta proteins, thymic stromal lymphopoietins (“TSLP”), RANK ligand (“OPGL”), c-kit, TNF receptors, including TNF Receptor Type 1, TRAIL-R2, angiopoietins, and biologically active fragments or analogs or variants of any of the foregoing.

Exemplary proteins and antibodies include Activase® (Alteplase); Aranesp® (Darbepoetin-alfa), Epogen® (Epoetin alfa, or erythropoietin); Avonex® (Interferon β-Ia); Bexxar® (Tositumomab); Betaseron® (Interferon-β); Campath® (Alemtuzumab); Dynepo® (Epoetin delta); Velcade® (bortezomib); MLN0002 (anti-α4β7 mAb); MLN1202 (anti-CCR2 chemokine receptor mAb); Enbrel® (etanercept); Eprex® (Epoetin alfa); Erbitux® (Cetuximab); Genotropin® (Somatropin); Herceptin®(Trastuzumab); Humatrope® (somatropin [rDNA origin] for injection); Humira® (Adalimumab); Infergen® (Interferon Alfacon-1); Natrecor® (nesiritide); Kineret® (Anakinra), Leukine® (Sargamostim); LymphoCide® (Epratuzumab); Benlysta™ (Belimumab); Metalyse® (Tenecteplase); Mircera® (methoxy polyethylene glycol-epoetin beta); Mylotarg® (Gemtuzumab ozogamicin); Raptiva® (efalizumab); Cimzia® (certolizumab pegol); Soliris™ (Eculizumab); Pexelizumab (Anti-C5 Complement); MEDI-524 (Numax®); Lucentis® (Ranibizumab); Edrecolomab (Panorex®); Trabio® (lerdelimumab); TheraCim hR3 (Nimotuzumab); Omnitarg (Pertuzumab, 2C4); Osidem® (IDM-I); OvaRex® (B43.13); Nuvion® (visilizumab); Cantuzumab mertansine (huC242-DMI); NeoRecormon® (Epoetin beta); Neumega® (Oprelvekin); Neulasta® (Pegylated filgastrim, pegylated G-CSF, pegylated hu-Met-G-CSF); Neupogen® (Filgrastim); Orthoclone OKT3® (Muromonab-CD3), Procrit®(Epoetin alfa); Remicade® (Infliximab), Reopro® (Abciximab), Actemra® (anti-IL6 Receptor mAb), Avastin® (Bevacizumab), HuMax-CD4 (zanolimumab), Rituxan® (Rituximab); Tarceva® (Erlotinib); Roferon-A®-(Interferon alfa-2a); Simulect® (Basiliximab); Stelara™ (Ustekinumab); Prexige® (lumiracoxib); Synagis® (Palivizumab); 146B7-CHO (anti-IL15 antibody, see U.S. Pat. No. 7,153,507), Tysabri® (Natalizumab); Valortim® (MDX-1303, anti-B. anthracis Protective Antigen mAb); ABthrax™; Vectibix® (Panitumumab); Xolair® (Omalizumab), ETI211 (anti-MRSA mAb), IL-I Trap (the Fc portion of human IgGI and the extracellular domains of both IL-I receptor components (the Type I receptor and receptor accessory protein)), VEGF Trap (Ig domains of VEGFRI fused to IgGI Fc), Zenapax® (Daclizumab); Zenapax® (Daclizumab), Zevalin® (Ibritumomab tiuxetan), Zetia (ezetimibe), Atacicept (TACI-Ig), anti-α4β7 mAb (vedolizumab); galiximab (anti-CD80 monoclonal antibody), anti-CD23 mAb (lumiliximab); BR2-Fc (huBR3/huFc fusion protein, soluble BAFF antagonist); Simponi™ (Golimumab); Mapatumumab (human anti-TRAIL Receptor-1 mAb); Ocrelizumab (anti-CD20 human mAb); HuMax-EGFR (zalutumumab); M200 (Volociximab, anti-α5β1 integrin mAb); MDX-010 (Ipilimumab, anti-CTLA-4 mAb and VEGFR-I (IMC-18F1); anti-BR3 mAb; anti-C. difficile Toxin A and Toxin B C mAbs MDX-066 (CDA-I) and MDX-1388); anti-CD22 dsFv-PE38 conjugates (CAT-3888 and CAT-8015); anti-CD25 mAb (HuMax-TAC); anti-CD3 mAb (NI-0401); Adecatumumab (MT201, anti-EpCAM-CD326 mAb); MDX-060, SGN-30, SGN-35 (anti-CD30 mAbs); MDX-1333 (anti-IFNAR); HuMax CD38 (anti-CD38 mAb); anti-CD40L mAb; anti-Cripto mAb; anti-CTGF Idiopathic Pulmonary Fibrosis Phase I Fibrogen (FG-3019); anti-CTLA4 mAb; anti-eotaxinl mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); MEDI-545, MDX-1103 (anti-IFNα mAb); anti-IGFIR mAb; anti-IGF-IR mAb (HuMax-Inflam); anti-IL12/IL23p40 mAb (Briakinumab); anti-IL-23p19 mAb (LY2525623); anti-IL13 mAb (CAT-354); anti-IL-17 mAb (AIN457); anti-IL2Ra mAb (HuMax-TAC); anti-IL5 Receptor mAb; anti-integrin receptors mAb (MDX-O18, CNTO 95); anti-IPIO Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PDImAb (MDX-1 106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); NVS Antibody #1; and NVS Antibody #2.

Exemplary protein concentrations in the formulation may range from about 0.1 mg/ml to about 200 mg/ml, about 0.3 mg/ml to about 150 mg/ml, from about 0.1 mg/ml to about 70 mg/ml, from about 0.1 mg/ml to about 50 mg/ml, or from about 0.5 mg/ml to about 25 mg/ml, or alternatively from about 1 mg/ml to about 10 mg/ml. The concentration of protein will depend upon the end use of the pharmaceutical formulation and can be easily determined by a person of skill in the art. Particularly contemplated concentrations of protein are at least about 1.0, 1.5, 2.0, 2.5, 3.0, 3.5, 4.0, 5.0, 6.0, 7.0, 8.0, 9.0, 10.0, 15.0, 20.0, 25.0, 30.0, 35.0, or 40.0, or up to about 15.0, 20.0, 25.0, 30.0, 35.0, 40.0, 45.0, 50.0, 55.0, 60.0, 65.0, 70.0, 75.0, 80.0, 85.0, 90.0, 95.0, 100.0, 105.0, 110.0, 115.0, 120.0, 125.0, 130.0, 140.0, 150.0, 180.0, 190.0 and 200.00 mg/ml and including all values in between.

As used herein, “pharmaceutical formulation” is a sterile composition of a pharmaceutically active drug, such as a biologically active protein, that is suitable for parenteral administration (including but not limited to intravenous, intramuscular, subcutaneous, aerosolized, intrapulmonary, intranasal or intrathecal) to a patient in need thereof and includes only pharmaceutically acceptable excipients, diluents, and other additives deemed safe by the Federal Drug Administration or other foreign national authorities. Pharmaceutical formulations include liquid, e.g. aqueous, solutions that may be directly administered, and lyophilized powders which may be reconstituted into solutions by adding a diluent before administration. Specifically excluded from the scope of the term “pharmaceutical formulation” are compositions for topical administration to patients, compositions for oral ingestion, and compositions for parenteral feeding.

“Shelf life”, as used herein, means that the storage period during which an active ingredient such as a therapeutic protein in a pharmaceutical formulation has minimal degradation (e.g., not more than about 2-3% degradation) when the pharmaceutical formulation is stored under specified storage conditions, for example, 2-8° C. Techniques for assessing degradation vary depending upon the identity of the protein in the pharmaceutical formulation. Exemplary techniques include size-exclusion chromatography (SEC)-HPLC to detect, e.g., aggregation, reverse phase (RP)-HPLC to detect, e.g. protein fragmentation, ion exchange-HPLC to detect, e.g., changes in the charge of the protein, mass spectrometry, fluorescence spectroscopy, circular dichroism (CD) spectroscopy, Fourier transform infrared spectroscopy (FT-IR), and Raman spectroscopy to detect protein conformational changes. All of these techniques can be used singly or in combination to assess the degradation of the protein in the pharmaceutical formulation and determine the shelf life of that formulation. The pharmaceutical formulations of the present invention preferably exhibit not more than about 2 to about 3% increases in degradation (e.g., fragmentation, aggregation or unfolding) over two years when stored at 2-8° C.

As used herein, “stable” formulations of biologically active proteins are formulations that exhibit reduced aggregation and/or reduced loss of biological activity of at least 5% upon storage at 2-8° C. for at least 2 years compared with a control formula sample, or alternatively which exhibit reduced aggregation and/or reduced loss of biological activity under conditions of thermal stress, e.g. 52° C. for 7-8 days.

As used herein, “viscosity” is a fluid's resistance to flow, and may be measured in units of centipoise (cP) or milliPascal-second (mPa-s), where 1 cP=I mPa-s, at a given shear rate. Viscosity may be measured by using a viscometer, e.g., Brookfield Engineering Dial Reading Viscometer, model LVT. Viscosity may be measured using any other methods and in any other units known in the art (e.g. absolute, kinematic or dynamic viscosity), understanding that it is the percent reduction in viscosity afforded by use of the excipients described by the invention that is important. Regardless of the method used to determine viscosity, the percent reduction in viscosity in zwitterion excipient formulations versus control formulations will remain approximately the same at a given shear rate.

As used herein, a formulation containing an amount of an excipient effective to “reduce viscosity” (or a “viscosity-reducing” amount or concentration of such excipient) means that the viscosity of the formulation in its final form for administration (if a solution, or if a powder, upon reconstitution with the intended amount of diluent) is at least 5% less than the viscosity of an appropriate control formulation, such as those, for example, containing polyols and exemplified herein. Excipient-free control formulations might also be used but may not always be the most appropriate control formulation because such a formulation may not be implementable as a therapeutic formulation due to hypotonicity, for instance. Formulations containing zwitterion excipients are useful because they may be used to create an isotonic formulation without contributing to viscosity increases.

Similarly, a “reduced viscosity” formulation is a formulation that exhibits reduced viscosity compared to a control formulation.

Protein therapeutics often need to be given at high concentration but for injection a smaller volume is necessary which can result in increased viscosity of the solution. When large doses of therapeutic protein are to be administered in a small volume of liquid (such as for injection), it is highly desirable to provide formulations with high concentrations of protein that do not exhibit the increased viscosity typically seen with such high protein concentrations.

High viscosity formulations are difficult to handle during manufacturing, including at the bulk and filling stages. High viscosity formulations are also difficult to draw into a syringe and inject, often necessitating use of lower gauge needles which can be unpleasant for the patient. The addition of taurine, theanine, sarcosine, citrulline, betaine or mixtures thereof, to solutions of biologically active protein unexpectedly reduced the viscosity of high concentration protein solutions.

The use of an excipient selected from the group consisting of taurine, theanine, sarcosine, citrulline, betaine and mixtures thereof, permits a higher concentration of therapeutic proteins to be used in the formulation without a concomitant increase in viscosity. Thus, the invention provides a method for stabilizing or reducing viscosity of protein formulations by adding an excipient selected from the group consisting of combining taurine, theanine, sarcosine, citrulline, betaine and mixtures thereof, in an amount effective to reduce viscosity. The invention also provides reduced viscosity formulations of therapeutic proteins, including antibodies, containing effective amounts or concentrations of an excipient selected from the group consisting of combining taurine, theanine, sarcosine, citrulline, betaine and mixtures thereof. Also contemplated are methods of screening one or more formulations, each containing different concentrations of taurine, theanine, sarcosine, citrulline, betaine and mixtures thereof, to identify suitable or optimal concentrations that reduce viscosity. Further provided are methods of preparing a lyophilized powder from reduced viscosity solution formulations of the invention, and methods of reconstituting the lyophilized powders of the invention via addition of a sterile diluent.

Thus, the present invention provides pharmaceutical formulations containing biologically active polypeptides and viscosity-reducing concentrations of excipients selected from the group consisting of taurine, theanine, sarcosine, citrulline, betaine and mixtures thereof. The reduction in viscosity is at least about 10-70% versus control formulations. In one embodiment the reduction in viscosity ranges from about 10-30%. In other exemplary embodiments, the reduction in viscosity is at least 5%, 10%, 15%, 20%, 25%, 30%, 35%, 40%, 45%, 50%, 55%, 60%, or 65%.

Formulations of the invention may optionally include pharmaceutically acceptable salts, buffers, surfactants, other excipients, carriers, diluents, and/or other formulation agents.

Exemplary pharmaceutically acceptable buffers include acetate (e.g. sodium acetate), succinate (such as sodium succinate), glutamic acid, glutamate, gluconate, histidine, citrate or other organic acid buffers. Exemplary buffer concentration can be from about 1 mM to about 200 mM, or from about 10 mM to about 60 mM, depending, for example, on the buffer and the desired tonicity (e.g. isotonic, hypertonic or hypotonic) of the formulation. Exemplary pHs include from about 4.5 to about 6.5, or from about 4.8 to about 5.5, or from about 4 to 6, or about 5 to 5.5, or about 5, greater than about 5, greater than about 5.5, greater than about 6, or greater than about 6.5.

Suitable diluents, other excipients, or carriers and other agents include, but are not limited to, antioxidants, coloring, flavoring and diluting agents, emulsifying agents, suspending agents, solvents, fillers, bulking agents, buffers, vehicles, diluents and/or pharmaceutical adjuvants. For example, a suitable vehicle may be, physiological saline solution, citrate buffered saline, or artificial CSF, possibly supplemented with other materials common in compositions for parenteral administration. Neutral buffered saline or saline mixed with serum albumin are further exemplary vehicles. Those skilled in the art would readily recognize a variety of buffers that could be used in the compositions, and dosage forms used in the invention. Typical buffers include, but are not limited to pharmaceutically acceptable weak acids, weak bases, or mixtures thereof. Exemplary buffer components are water soluble materials such as phosphoric acid, tartaric acids, lactic acid, succinic acid, citric acid, acetic acid, ascorbic acid, aspartic acid, glutamic acid, or salts thereof. Exemplary salts include inorganic and organic acids, or bases such as metals or amines, in exemplary concentrations such as about 50-200 mM, or 100-200 mM, or about 100 mM, or about 150 mM.

Other excipients or stabilizers may also be included, for example, sugars (e.g., sucrose, glucose, trehalose, fructose, xylose, mannitose, fucose), polyols (e.g., glycerol, mannitol, sorbitol, glycol, inositol), amino acids or amino acid derivatives, or surfactants (e.g., polysorbate, including polysorbate 20, or polysorbate 80, or poloxamer, including poloxamer 188). Exemplary concentrations of surfactant may range from about 0.001% to about 0.5%, or from about 0.003% to about 0.2%. Preservatives may also be included, such as benzyl alcohol, phenol, m-cresol, chlorobutanol or benzethonium Cl, e.g. at concentrations ranging from about 0.1% to about 2%, or from about 0.5% to about 1%.

One or more other pharmaceutically acceptable carriers, excipients or stabilizers such as those described in Remington's Pharmaceutical Sciences 21st edition, Osol, A. Ed. (2005) may be included in the formulation provided that they do not adversely affect the desired characteristics of the formulation.

The concentration of the therapeutic protein, such as an antibody, in the formulation will depend upon the end use of the pharmaceutical formulation and can be easily determined by a person of skill in the art.

Therapeutic proteins that are antagonists are frequently administered at higher concentrations than those that are agonists. Particularly contemplated high concentrations of therapeutic proteins (without taking into account the weight of chemical modifications such as pegylation), including antibodies, are at least about 70, 80, 90, 100, 110, 120, 130, 140, 150, 175, 200, 250, 300, 350, 400, 450, or 500 mg/ml, and/or less than about 250, 300, 350, 400, 450 or 500 mg/ml. Exemplary high concentrations of therapeutic proteins, such as antibodies, in the formulation may range from at least about 100 mg/ml to about 500 mg/ml. Other protein concentrations (without taking into account the weight of chemical modifications such as pegylation), are also contemplated, e.g., at least about 1, 5, 10, 20, 30, 35, 40, 45, 50, 55, 60, 65 or 70 mg/ml. The invention particularly contemplates formulations and methods in which the concentration of therapeutic protein results in a viscosity of at least 6, 8, 10, 12, 14, 16, 18, 20, 25, 30 cP or higher and the inclusion of combining taurine, theanine, sarcosine, citrulline, betaine and combinations thereof results in the reduction of the viscosity by 5% or greater. For example, a solution with a viscosity of about 20 cP may be difficult to inject with a standard 27 gauge needle. All references to mg/ml concentration of therapeutic protein, weight of therapeutic protein (mg) or molecular weight of therapeutic protein (kD) herein mean the respective weight of the proteinaceous part of the therapeutic protein, excluding any non-proteinaceous modifications.

The present invention provides a method of reducing the viscosity of and/or improving stability of a liquid pharmaceutical formulation of a therapeutic protein, by combining the therapeutic protein and a viscosity-reducing amount of an excipient selected from the group consisting of taurine, theanine, sarcosine, citrulline, betaine and mixtures thereof.

In exemplary embodiments, the therapeutic protein is at a high protein concentration as described above. In some embodiments, the reduction in viscosity is at least about 5%, 10%, 15%, 20%, 25%, 30%, 35%, 40%, 45%, 50%, 55%, 60%, 65% or 70% compared to control formulations

In another aspect, the invention provides liquid solutions comprising a therapeutic protein and an excipient selected from the group consisting of taurine, theanine, sarcosine, citrulline, betaine and mixtures thereof, wherein the formulations exhibit reduced viscosity relative to control formulations. In exemplary embodiments, the therapeutic protein is at a high protein concentration as described above. In some embodiments, the excipient is present at a viscosity-reducing (weight:volume) concentration. Any of these excipients can be used at concentrations up to their solubility limit. Such solutions may further comprise a sugar or other polyol such as sucrose or sorbitol, in an amount effective to further improve stability, reduce aggregation, and/or make the formulation isotonic, without significantly increasing viscosity.

In exemplary embodiments, the concentration of an excipient selected from the group consisting of taurine, theanine, sarcosine, citrulline, betaine and mixtures thereof, is at least about 10 μM to about 300 mM, or at least about 10 μM to about 650 mM, or at least about 1 μM to about 750 mM. In exemplary embodiments the concentration of the excipient is at least about 1, 5, 10, 50, 100, 200, 250, 300, 350, 400, 500, 600, 640, 650, 700 or 750 mM or greater. Other exemplary embodiments include concentrations of excipients effective to make the formulation isotonic, without significantly increasing viscosity. Exemplary concentrations include those at least about 200 mM or greater, in further embodiments the amounts are at least about 600 mM or greater. In further exemplary embodiments the concentration of taurine is at least about 200 mM or greater, in other embodiments the concentration is at least about 600 mM or greater.

In another aspect, the invention provides lyophilized protein formulations comprising a therapeutic protein and an excipient selected from the group consisting of taurine, theanine, sarcosine, citrulline, betaine and mixtures thereof, wherein upon reconstitution with the recommended amount of diluent, the formulations exhibit reduced viscosity relative to control formulations. In exemplary embodiments, the therapeutic protein is at a high protein concentration as described above. In some embodiments, the excipient is present at an amount effective to reduce viscosity upon reconstitution with diluent (weight:weight concentration). Such formulations may further comprise a sugar or other polyol such as sucrose or sorbitol, in an amount effective to further improve stability, reduce aggregation, and/or make the formulation isotonic, without significantly increasing viscosity.

In exemplary embodiments, the concentration of an excipient selected from the group consisting of taurine, theanine, sarcosine, citrulline, betaine and mixtures thereof, is at least about 1 μg per mg therapeutic protein, up to about 1.0 mg per mg therapeutic protein. In some embodiments, the concentration of excipient is at least about 1, 10, 50, 100, 150, 200, 250, 300, 350, 400, 450, 500 or 550 μg per mg therapeutic protein. In other exemplary embodiments, the concentration of excipient is up to about 600, 650, 700, 750, 800, 850, 900, 950 or 1000 μg per mg therapeutic protein.

In yet another embodiment, the present invention provides a method of preventing self-association of proteins in liquid formulations by using taurine, theanine, sarcosine, betaine, citrulline or mixtures thereof, as excipients in any of the amounts or concentrations described herein. Formulations with improved stability (e.g., reduced aggregation) and shelf-life are also provided.

The invention also provides a kit comprising a liquid protein formulation of the invention, and instructions for its administration, optionally with a container, syringe and/or other administration device. The invention further provides a kit comprising a lyophilized protein formulation of the invention, optionally in a container, and instructions for its reconstitution and administration, optionally with a vial of sterile diluent, and optionally with a syringe or other administration device. Exemplary containers include vials, tubes, bottles, single or multi-chambered pre-filled syringes, or cartridges. Exemplary administration devices include syringes, with or without needles, infusion pumps, jet injectors, pen devices, transdermal injectors, or other needle-free injector, or an aerosolization device for nasal or pulmonary delivery.

In another aspect, a method is provided for screening for a viscosity-reducing concentration of an excipient comprising the steps of: (1) assessing the viscosity of a first solution comprising a first concentration of excipient(s) selected from the group consisting of taurine, theanine, sarcosine, citrulline, betaine and mixtures thereof, and a therapeutic protein, such as an antibody, (2) assessing the viscosity of a second solution comprising a different second concentration of the excipient(s) and the therapeutic protein, and (3) determining that the first concentration of excipient(s) is more viscosity-reducing than the second concentration of excipient if the first solution is less viscous. Viscosity can be determined, e.g., using a Brookfield RV-DVIII Rheometer which is stabilized at 250 C with a circulating temperature bath. Five hundred microliters of sample is pipetted into the rheometer and the rpm adjusted for percentage torque values between 10-80%. The samples are allowed to stabilize at that range and data points are collected.

Similar methods are provided for screening for an aggregation-reducing or stabilizing concentration of an excipient.

Stability can be assessed in many ways, including monitoring conformational change over a range of temperatures (thermostability) and/or time periods (shelf-life) and/or after exposure to stressful handling situations (e.g. physical shaking). Stability of formulations containing varying concentrations of formulation components can be measured using a variety of methods. For example, the amount of protein aggregation can be measured by visual observation of turbidity, by measuring absorbance at a specific wavelength, by size exclusion chromatography (in which aggregates of a protein will elute in different fractions compared to the protein in its native active state), HPLC, or other chromatographic methods. Other methods of measuring conformational change can be used, including using differential scanning calorimetry (DSC), e.g. to determine the temperature of denaturation, or circular dichroism (CD), which measures the molar ellipticity of the protein. Fluorescence can also be used to analyze the composition. Fluorescence encompasses the release or absorption of energy in the form of light or heat, and changes in the polar properties of light. Fluorescence emission can be intrinsic to a protein or can be due to a fluorescence reporter molecule. For example, ANS is a fluorescent probe that binds to the hydrophobic pockets of partially unfolded proteins. As the concentration of unfolded protein increases, the number of hydrophobic pockets increases and subsequently the concentration of ANS that can bind increases. This increase in ANS binding can be monitored by detection of the fluorescence signal of a protein sample. Other means for measuring stability can be used and are well known to persons of skill in the art.

The invention will be more fully understood by reference to the following examples which detail exemplary embodiments of the invention. They should not, however, be construed as limiting the scope of the invention. All citations throughout the disclosure are hereby expressly incorporated by reference.

### EXAMPLES

### Example 1

The effects of protein concentration on the viscosity of antibody formulations containing an IgG2 monoclonal antibody, at pH 5-5.2 were studied. First, 70 mg/ml of the antibody formulated in 10 mM Sodium Acetate 9% Sucrose pH 5.20 was dialyzed against 4 liters of 20 mM Na Acetate pH 4.7. Dialysis was carried out at 4° C. overnight using 10,000 MWCO SnakeSkin® pleated dialysis tubing (Thermo Fisher Scientific, Rockford, Ill.). Next, the antibody was filtered through a 0.22 μm cellulose acetate filter. The protein was concentrated by subjecting to centrifugation with 30,000 MWCO Amicon® Ultracel centrifugal filter (Millipore, Billerica, Mass.) in an Allegra X-12R Centrifugue (Beckman Coulter, Brea, Calif.). Protein concentration was determined by Agilent 8453 UV/Vis Spectrophotometer (Santa Clara, Calif.) and the concentration was adjusted to 200 mg/ml, pH 5.20. Test samples were prepared by formulating the concentrated antibody solution to 200 mM with sarcosine, theanine, betaine, taurine, L-citrulline, sucrose, serine, glycine, alanine, creatine (Sigma Aldrich, St. Louis, Mo.). The control was a non-excipient containing formulation. Viscosities were measured using a RV-DV III+ Programmable Rheometer (Brookfield Engineering, Middleboro, Mass.) stabilized at 25° C. with a circulating temperature bath and calibrated with a mineral oil standard at 29.24 cP before each set of samples was run. Sample volumes of 0.5 ml were tested for each measurement using a CPE-40 cone and matching cup. Three data points were collected in the low (25 rpm), middle (50 rpm) and high torque (100 rpm) ranges.

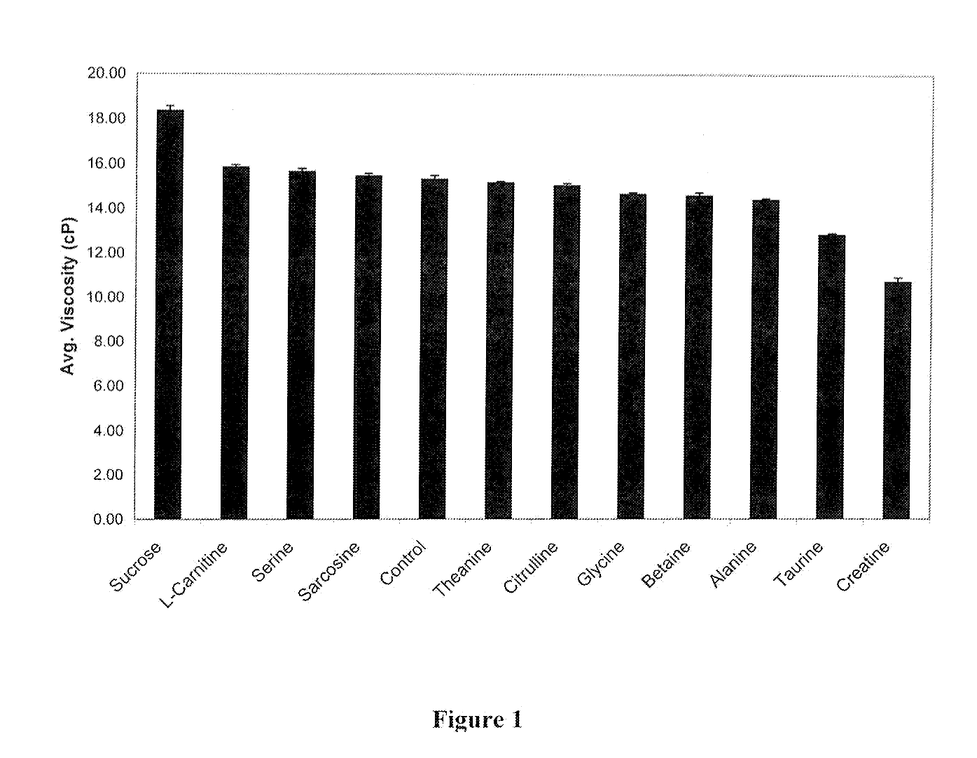

FIG. 1 shows the effects of the various excipients on the viscosity of the antibody solution. The data show that the tested excipients have reduced viscosity relative to the sucrose containing sample. For citrulline, sarcosine, betaine, and theanine this decrease is on the order of 15-20% while for taurine the decrease is approximately 30%.

### Example 2

A concentrated antibody solution (200 mg/ml) was created as described in Example 1. Test samples were prepared by formulating the concentrated antibody solution with sucrose, citrulline or theanine at concentrations of 50, 100 and 200 mM. Viscosities were measured as described in Example 1.

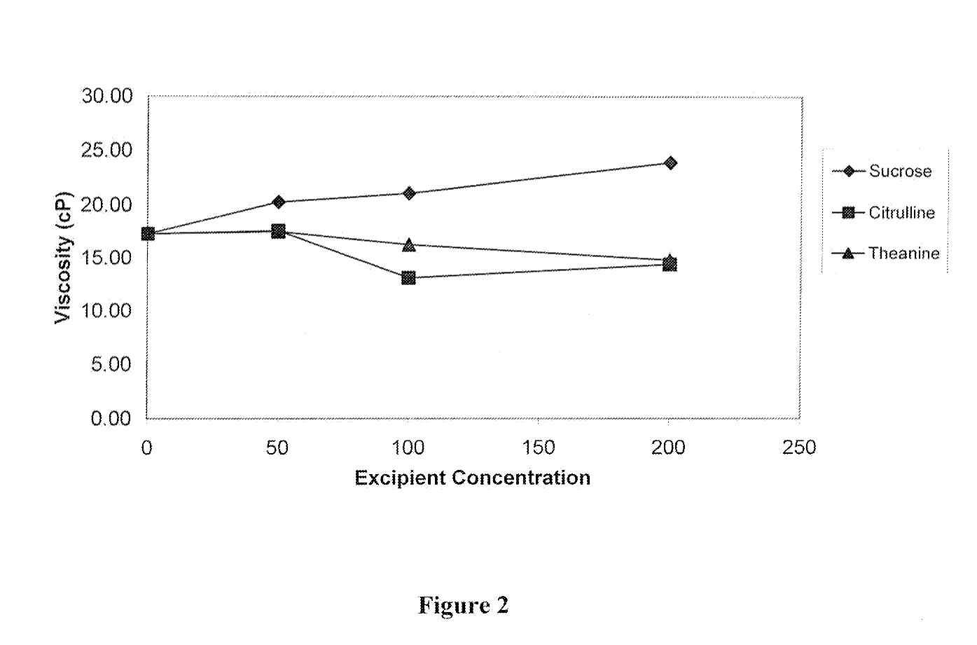

FIG. 2 shows the effects of varying concentrations of citrulline and theanine on the viscosity of the antibody solution. This data shows that in contrast to sucrose containing formulations which had increased viscosity with increasing concentrations of sucrose, the excipient containing formulation viscosity decreased with increasing concentration of excipient.

### Example 3

The effect of taurine, theanine, sarcosine, citrulline and betaine on protein stability was assessed by addition to the antibody formulation. A concentrated antibody formulation was created as described in Example 1 and test samples were prepared by formulating the concentrated antibody to 100 mM with taurine, theanine, sarcosine, citrulline, betaine, sucrose, sorbitol, B-alanine, L-carnitine, creatine, serine, L-alanine, or glycine. The samples were sterile filtered and filled in 3 cc glass vials and stored for 3 months at 25° C. Samples were analyzed by Size-Exclusion Chromatography (SEC-HPLC) using an Agilent 1100 HPLC (Santa Clara, Calif.). TSKgel G3000 SWXL 7.8 mm×30 cm column was used. Mobile phase was 80 mM sodium phosphate, 300 mM sodium perchlorate 10% (v/v) isopropyl alcohol, pH 7.2. Flow rate was 0.5 mL/minute, UV detection was at 215 nm.

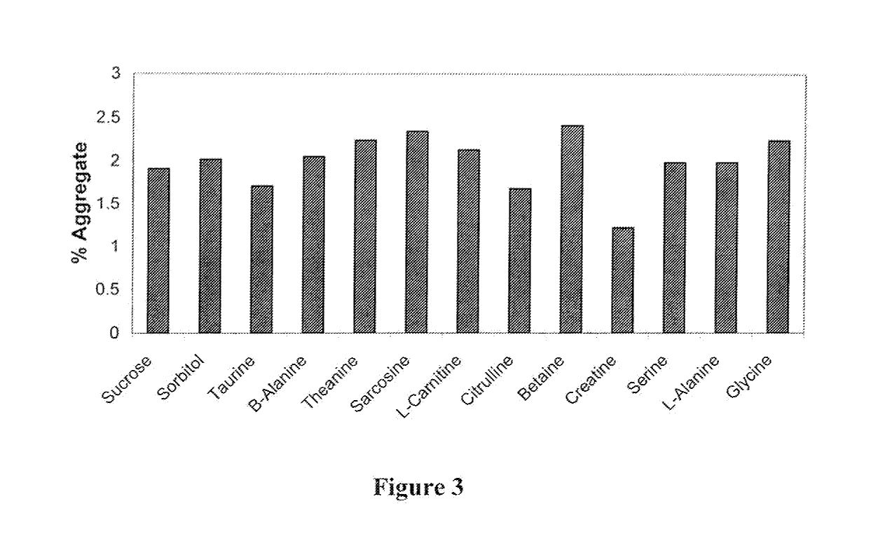

FIG. 3 shows the effects of varying concentrations of the excipients on the viscosity of the antibody solution. The data show that the excipient formulations have comparable stability to polyol formulations with respect to aggregate formation. However, there were significant improvements in viscosity shown for these excipients, with no compromise to the stability of the antibody.

### Example 4

A concentrated antibody solution (215 mg/ml) was prepared as described in Example 1. Test samples were prepared by formulating the concentrated IgG2 antibody solution with either L-citrulline or sucrose at 275 mM. Viscosities were measured as described in Example 1.

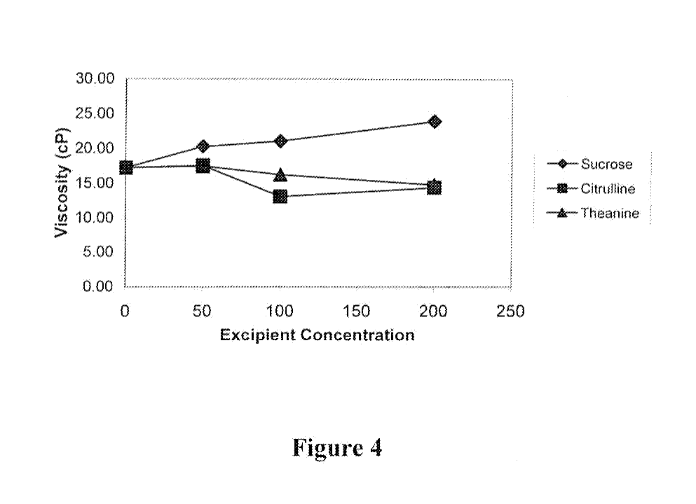

FIG. 4 shows the viscosity is ˜13% lower for the L-citrulline formulation compared to the sucrose formulation.

### Example 5

The effects of taurine on the viscosity of antibody formulations containing an IgG2 monoclonal antibody were studied. Three samples (2 ml) of an IgG2 antibody preparation (70 mg/ml) were concentrated to ˜0.7 ml using a Centricon™ 30 kd MWCO concentrator (Millipore, Billerica, Mass.), protein concentration determined using UV-Vis. “Taurine 4.8” received 2.0 ml of taurine buffer (10 mM glutamate, 260 mM taurine, pH 3.8), “Taurine 5.0” received 2.0 ml taurine buffer (10 mM glutamate, 260 mM taurine, pH 4.3). The third formulation tested was 10 mM sodium acetate 9% sucrose pH 5.20 “Sucrose”. The samples were concentrated by ultracentrifugation for an additional 30 minutes. The process was repeated 2 additional times. The supernatants were collected for viscosity and stability evaluation and protein concentration was determined by UV-Vis. The concentrated antibody solutions ranged from 180-189 mg/ml, see Table 1.

TABLE 1
Concentration and pH for antibody solutions after ultracentrifugation.

| mAb | | | |
|---|---|---|---|
| formulation | Sucrose | Taurine 4.8 | Taurine 5.0 |
| Buffer pH | 5.2 | 3.8 | 4.3 |
| Final mAb pH | 5.2 | 4.79 | 5.01 |
| (mg/mL) | 180.0 | 188.88 | 181.8 |

Viscosities were measured using a RV-DV III+ Programmable Rheometer (Brookfield Engineering, Middleboro, Mass.) stabilized at 25° C. with a circulating temperature bath. Spindle speed ranged from 15 to 125 rpm with 10 rpm per increment. Data collection was carried out with Rehoclac software, version 2.7. At each shear rate, a wait time of 30 seconds was allowed to equilibrate the system before the first reading and four readings of 10 second intervals were made. Each data point is the average of four readings.

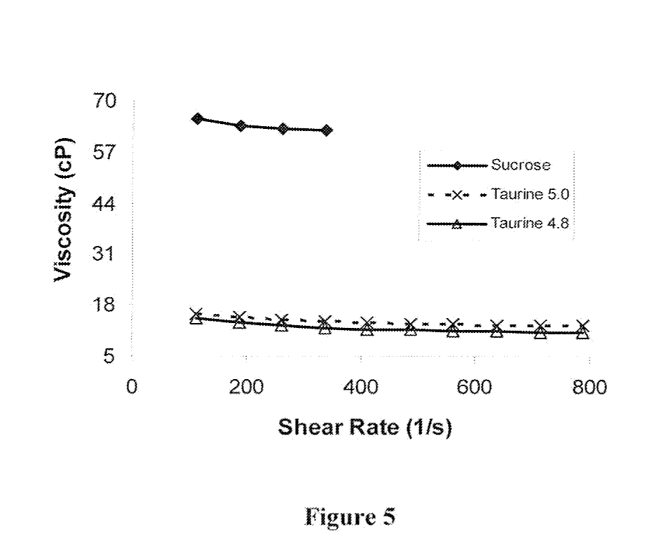

FIG. 5 shows the effect of taurine on viscosity. After centrifugation concentrations, the antibody pH increased to 4.8 and 5.0 as can be seen in Table 1. The pH of the samples is comparable to the acetate formulation containing sucrose (pH 5.2) suggesting that taurine, rather than pH, contributes significantly to lower viscosity in the antibody samples. The taurine formulations also took less than half the time to centrifuge compared to the sucrose containing formulation.

### Example 6

The effect of taurine and sarcosine on protein stability was assessed by addition to the antibody formulation. An IgG2 antibody formulation (70 mg/ml) was dialyzed into various excipient formulations, (Table 2) and concentrated to 150 mg/ml. The samples were sterile filtered and filled in 3 cc glass vials and stored for 6 months at 37° C. Samples were analyzed by Size-Exclusion Chromatography using an Agilent 1100 HPLC with TSKgel G3000 SWXL column. Mobile phase was 100 mM sodium phosphate, 150 mM sodium chloride, pH 7.0. Flow rate was 0.5 mL/minute, UV detection was at 280 nm.

TABLE 2
Sample formulations

| Sample | Formulation |
|---|---|
| Glycine | 10 mM Glutamic acid, 240 mM Glycine, pH 4.6, |
| | 0.01% Tween 20 |
| Sarcosine | 10 mM Glutamic acid, 240 mM Sarcosine, pH 4.8, |
| | 0.01% Tween 20 |
| Proline | 10 mM NaAcetate, 3% Proline, pH 4.8, |
| | 0.01% Tween 20 |
| Taurine | 10 mM Glutamic acid, 260 mM Taurine, pH 4.8, |
| | 0.01% Tween 20 |
| Sucrose | 10 mM Glutamate, 9.0% Sucrose, 0.01% Tween 20, |
| | pH 4.91 |

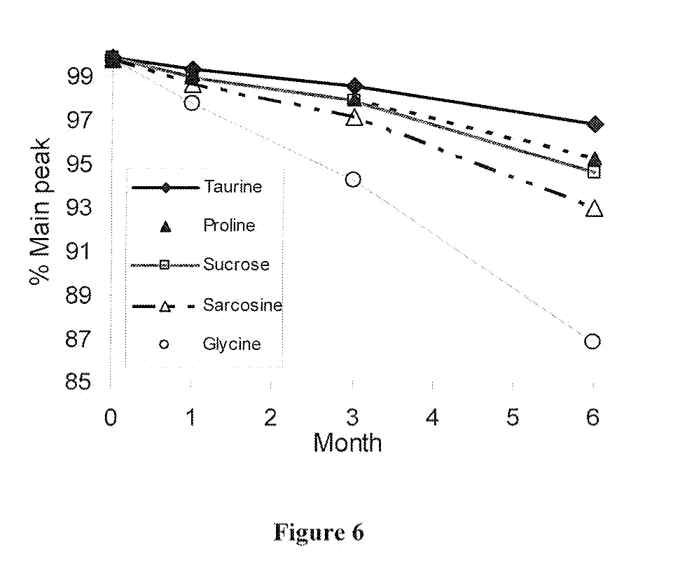

FIG. 6 shows the effects of taurine and sarcosine on thermally induced aggregation of a concentrated antibody formulation. The taurine containing formulation shows the highest stability at 37° C. after 6 months, yet maintains the lowest viscosity, especially at 4° C. (Table 3).

TABLE 3
Viscosity of various excipient formulations at 4° C. and 25° C.

Viscosity (cP)

| Excipient | 4° C. | 25° C. |
|---|---|---|
| Glycine | 16.60 | 6.29 |
| Sarcosine | 16.82 | 6.41 |
| Proline | 18.93 | 6.89 |
| Taurine | 14.27 | 6.05 |
| Sucrose | 29.13 | 10.47 |

### Example 7

An IgG2 antibody preparation (70 mg/ml) was concentrated using ultrafiltration and diafiltration to ˜90 mg/ml with 5 diafiltration volumes of Buffer A (10 mM glutamate, 0.5% sucrose, pH 4.2) or Buffer B (10 mM glutamate, 0.5% sucrose, pH 5.2). The concentration of both antibody preparations following UF/DF was ˜90 mg/ml. The samples were sterile filtered and 1.25 ml was filled in 3 cc glass vials and lyophilized. At room temperature, the Iyo cakes from Buffer A and Buffer B were formulated with various excipients, Table 3. Viscosity was determined as described in Example 5.

TABLE 4
Sample formulations

| | |
|---|---|
| Sample | Formulation |

Buffer A

| | |
|---|---|
| Taurine A (Tau A) | 10 mM glutamate, 260 mM taurine, pH 3.8 |
| Creatinine (CN) | 10 mM glutamate, 200 mM creatinine, pH 4.5 |

Buffer B

| | |
|---|---|
| TMAO | 10 mM glutamate, 260 mM TMAO, pH 4.5 |
| | (TMAO Sigma Aldrich) |
| Sarcosine (Sar) | 10 mM glutamate, 260 mM sarcosine, pH 4.5 |
| Taurine B (Tau B) | 10 mM glutamate, 260 mM taurine, pH 3.8 |

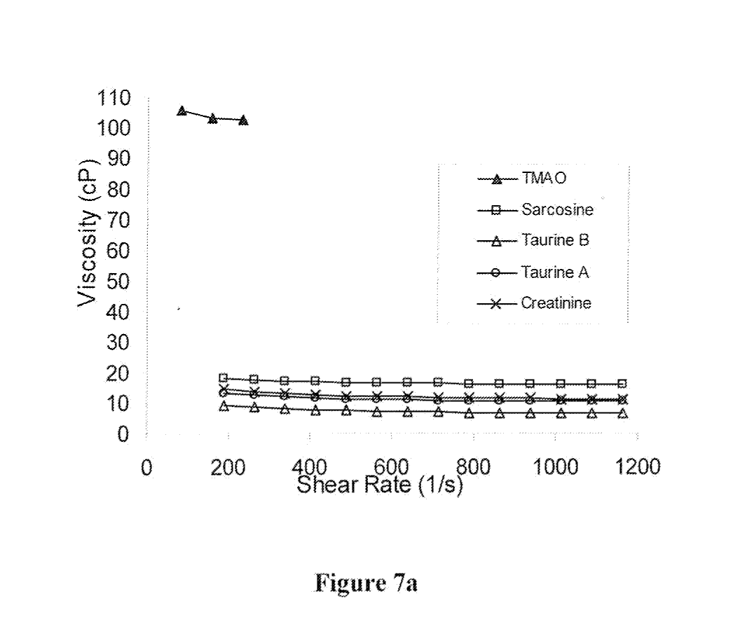

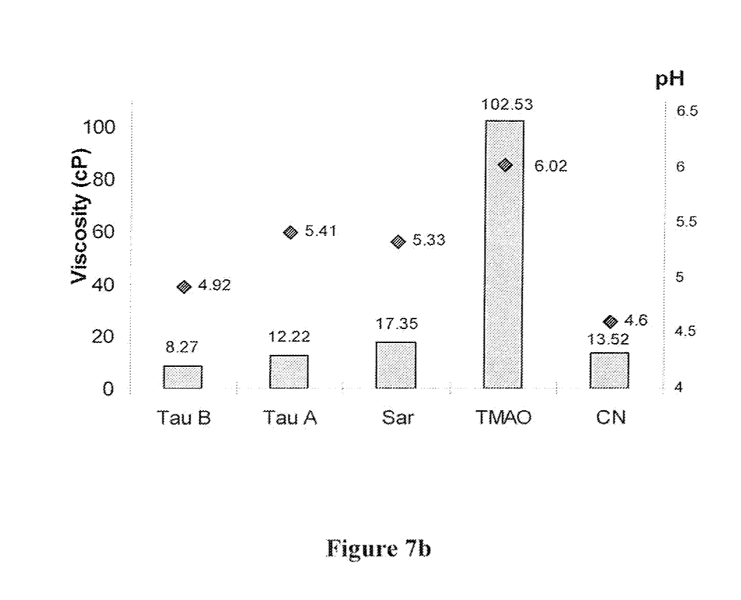

FIG. 7(a) shows viscosity comparison of the high concentration antibody formulations as a function of shear rate. FIG. 7(b) shows a comparison of excipients and pH effects on lowering viscosity of the concentrated antibody solution. The viscosity values are shown as a bar graph on the left axis at a specific shear rate. The corresponding formulation pHs are shown in scattered plot at the right axis.

A side-by-side comparison of low pH formulations containing 200 mM carnitine, creatinine and taurine was done. Lyphilized Buffer B samples from above were reconstituted as shown in Table 5.

TABLE 5
Sample formulations

| Sample | Formulation |
|---|---|
| Creatinine | 10 mM Glutamate, 200 mM creatinine, pH 2.8 |
| Carnitine | 10 mM Glutamate, 200 mM carnitine, pH 2.8 |
| Taurine | 10 mM Glutamate, 200 mM taurine, pH 3.1 |

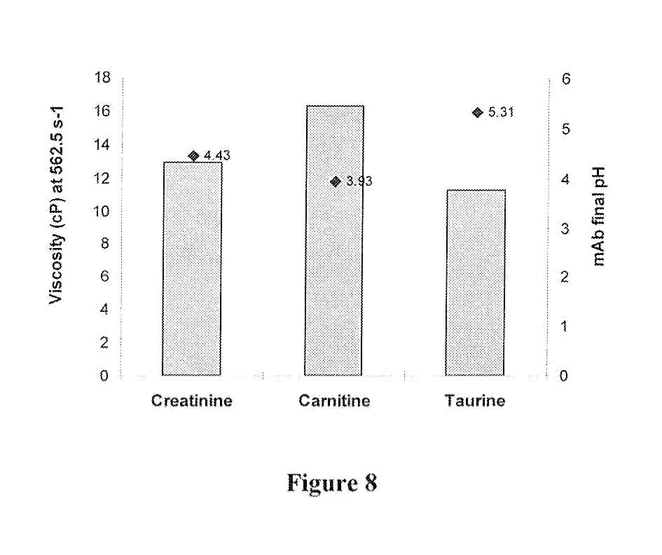

Viscosity of each sample was determined as described in Example 5. After reconstitution the pH of the taurine formulation pH rose to 5.31, higher than either creatinine or carnitine, but the viscosity of the taurine formulation remained lower than either the creatinine or carnitine formulations (FIG. 8). This suggests that taurine may be more effective than creatinine or carnitine in lowering viscosity of a concentrated antibody formulation.

What is claimed is:

1. A method for reducing the viscosity of a liquid pharmaceutical formulation comprising a therapeutic protein at a concentration of at least 70 mg/ml, comprising the step of combining the therapeutic protein with a viscosity-reducing concentration of an excipient selected from the group consisting taurine, theanine, sarcosine, citrulline, betaine and mixtures thereof.

2. The method of claim 1 wherein viscosity of the formulation is reduced by at least 5%.

3. The method of claim 1 wherein viscosity of the formulation is reduced by at least 30%.

4. A pharmaceutical formulation produced by the method of claim 1.

5. A pharmaceutical composition comprising a therapeutic protein at a concentration of at least 70 mg/mL, and an excipient selected from the group consisting of taurine, theanine, sarcosine, citrulline, betaine and mixtures thereof.

6. A pharmaceutical composition of claim 5, wherein the concentration of the excipient is from about 5 mM to about 700 mM.

7. A pharmaceutical composition of claim 6, wherein the concentration of the excipient is from about 200 mM to about 650 mM.

8. A pharmaceutical composition of claim 5 having a pH between about 4.0 to about 6.0.

9. A pharmaceutical composition of claim 8 having a pH of about 4.6 to about 5.2.

10. A method of preparing a lyophilized powder comprising the step of lyophilizing a pharmaceutical formulation comprising a therapeutic protein at a concentration of at least 70 mg/mL, and an excipient selected from the group consisting of taurine, theanine, sarcosine, citrulline, betaine and mixtures thereof.

11. A lyophilized powder comprising a therapeutic protein and an excipient selected from the group consisting of taurine, theanine, sarcosine, citrulline, betaine and mixtures thereof, wherein the excipient is present at a weight:weight concentration effective to reduce viscosity upon reconstitution with a diluent.

12. A lyophilized powder of claim 11 wherein the excipient is present at a concentration of between about 100 μg per mg therapeutic protein to about 1 mg per mg therapeutic protein.

13. A lyophilized powder of claim 12 wherein the excipient is present at a concentration between about 200 μg to about 500 μg per mg therapeutic protein.

14. A method for reconstituting a lyophilized powder of claim 11, comprising the step of adding a sterile aqueous diluent.

15. The method of claim 1 wherein the therapeutic protein is an antibody.

16. A pharmaceutical composition of claim 5 wherein the therapeutic protein is an antibody.

17. A lyophilized powder of claim 11 wherein the therapeutic protein is an antibody.
